(19) United States
(12) Patent Application Publication
(10) Pub. No.: US 2010/0113996 A1
(43) Pub. Date: May 6, 2010

(54) Epicondylitis clasp

(75) Inventors: Rony Bätz, Vogtlandisches Oberland (DE); Gerald Stier, Langenwertzendorf (DE)

Correspondence Address: Juan Carlos A. Marquez;c/o Stites & Harbison PLLC, 1199 North Fairfax Street, Suite 900, Alexandria, VA, 22314-1437, US

(73) Assignee: Bauerfeind AG

(21) Appl. No.: 12/588,930

(22) Filed: Nov. 3, 2009

(30) Foreign Application Priority Data
Nov. 5, 2008 (DE) 10 2008 055 867.2

(51) Int. Cl. A61F 5/00

(52) U.S. Cl. 602/20

(57) ABSTRACT

The invention relates to an epicondylitis clasp, wherein the holding part thereof, encompassing the arm, is tightenable by means of a steplessly adjustable securing strap, the inside of said epicondylitis clasp being provided with a pad for exerting pressure on a pressure-requiring area, said pad being in the form of an oblong pad attached to the holding part. A side of the holding part opposite the opening therein—said opening being bridgeable by the securing strap—is provided with a tab, wherein, with the holding part fitted on the arm, said tab covers the elbow joint and, by receiving the elbow, so encompasses the elbow joint that, when the holding part is fitted, the tab, which is guided by the elbow, serves as a positioning aid for positioning the holding part. The holding part can be fitted by means of the positioning aid to either the left or right arm and, for fitting to the left or right arm, is provided on its inside with a support for the pad, by means of which support the pad is removably attached to the holding part by securing means such that the holding part can be fitted on either the left or right arm with the pad in the therapeutically required position.

The invention relates to an epicondylitis clasp, wherein the holding part thereof, encompassing the arm, is tightenable by means of a steplessly adjustable securing strap, the inside of said epicondylitis clasp being provided with a pad for exerting pressure on a pressure-requiring area, said pad being in the form of an oblong pad attached to the holding part. Such a clasp is described and presented in DE 197 16 705 C1. The pressure-requiring area may be the epicondyle, which is the relevant area in the case of so-called tennis elbow (epicondylitis radialis humeri). Also in the case of so-called golfer's arm (epicondylitis ulnaris humeri), there is a need for an area of the arm opposite the epicondyle to be exposed to a pressure. These are examples of applications of therapeutically required sites which must be decided on a case-by-case basis by the attending physician. In order to make said clasp usable for both the left and right arm, the pad is removably attached to the clasp and is rotatable by means of a shaft perpendicular to the longitudinal direction of the pad, with the result that the clasp can be brought, with respect to its oblong form, into that position in which it exerts a pressure on the epicondyle on either the left or right arm.

The above-described epicondylitis clasp must, through its pad, exert a considerable pressure on the relevant area of the arm in order for there to be the desired therapeutic effect, wherein, when the clasp is fitted and the securing strap is tightened, the shaft of the pad is subjected to shear forces which impose a considerable load on the shaft, as a consequence of which the shaft can be damaged. In addition, it has become apparent that, upon being fitted, the clasp may possibly have been fitted on the arm either too high, too low or turned out of position and must then be moved again by the user in order to bring the clasp with its pad into the correct position, this likewise placing a considerable load on the shaft of the pad.

The object of the invention is to facilitate the fitting of the epicondylitis clasp for a multiplicity of applications, more particularly for tennis elbow and golfer's arm, in each case in the therapeutically correct area, more specifically on the left or right arm, and to guarantee the determined position also in the case of repeated fitting. The object of the invention is achieved in that a side of the holding part opposite the opening therein—said opening being bridgeable by the securing strap—is provided with a tab, wherein, with the holding part fitted on the arm, said tab covers the elbow joint and, by receiving the elbow, so encompasses the elbow joint that, when the holding part is fitted, the tab, which is guided by the elbow, serves as a positioning aid for positioning the holding part, wherein said holding part can be fitted by means of the positioning aid to either the left or right arm and, for fitting to the left or right arm, is provided on its inside with a support for the pad, by means of which support the pad is removably attached to the holding part by securing means such that the holding part can be fitted on either the left or right arm with the pad in the therapeutically required position with respect to the epicondyle.

With regard to the attachment of the pad in two possible opposite positions on the clasp, depending on whether said clasp is fitted to the left or right arm or is intended to act as treatment for tennis elbow or golfer's arm, there is in either case a secure fastening of the pad, which, having been attached, does not require any further special adjustment, wherein the tab, which grows out of the clasp, serves as a positioning aid in order to ensure that the epicondylitis clasp, which forms a functional unit with the tab, is always fitted in the correct location and requires no subsequent adjustment, this additionally guaranteeing the security of fastening of the pad. The combination of positioning aid by means of the tab and the optional possibility of attachment of the pad means that the epicondylitis clasp according to the invention is comprehensively suitable for all known applications of arm-related complaints, especially those caused by sporting activities, as a consequence of which the attending physician requires just one single suitably adaptable epicondylitis clasp for all such treatment needs.

In order to form the support, the tab can be advantageously provided with a hole which, when the clasp is fitted, accommodates the elbow. Alternatively, it is possible for the tab to be provided with a recess which then serves to accommodate and especially protect the elbow.

An advantageous means of securing the pad may consist of a hook-and-pile fastener forming the securing means. Alternatively, it is possible for the securing means to be in the form of locking pins and holes to receive said locking pins. The locking pins can be formed on the pad with the corresponding holes being formed on the clasp. Alternatively, it is possible for the locking pins to be formed on the clasp and for the holes to be formed on the pad.

In order to ensure that the correct area is treated when the epicondylitis clasp is fitted, it is advantageous for the pad to be of such length in the circumferential direction of the holding part as to extend beyond the pressure-requiring areas. This is advantageously accomplished by a design in which the pad consists of a plurality of juxtaposed individual pads.

In order to make it pleasant to wear the epicondylitis clasp, the holding part is advantageously of such design that its border is softer than its inner region.

An illustrative embodiment of the invention is presented in the drawings, in which:

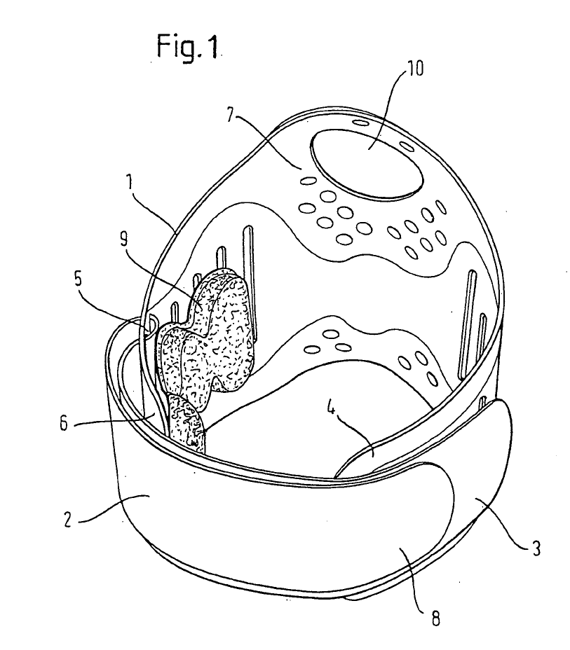

FIG. 1 shows the epicondylitis clasp with closed securing strap and a tab with a hole to accommodate the elbow;

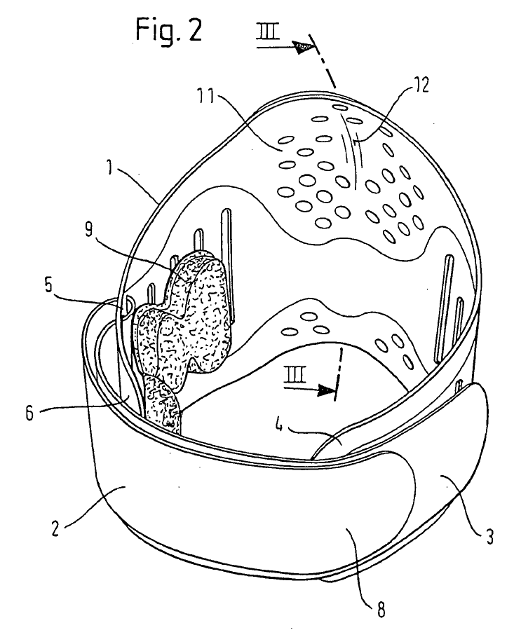

FIG. 2 shows the epicondylitis clasp from FIG. 1 and a tab with a recess to accommodate the elbow;

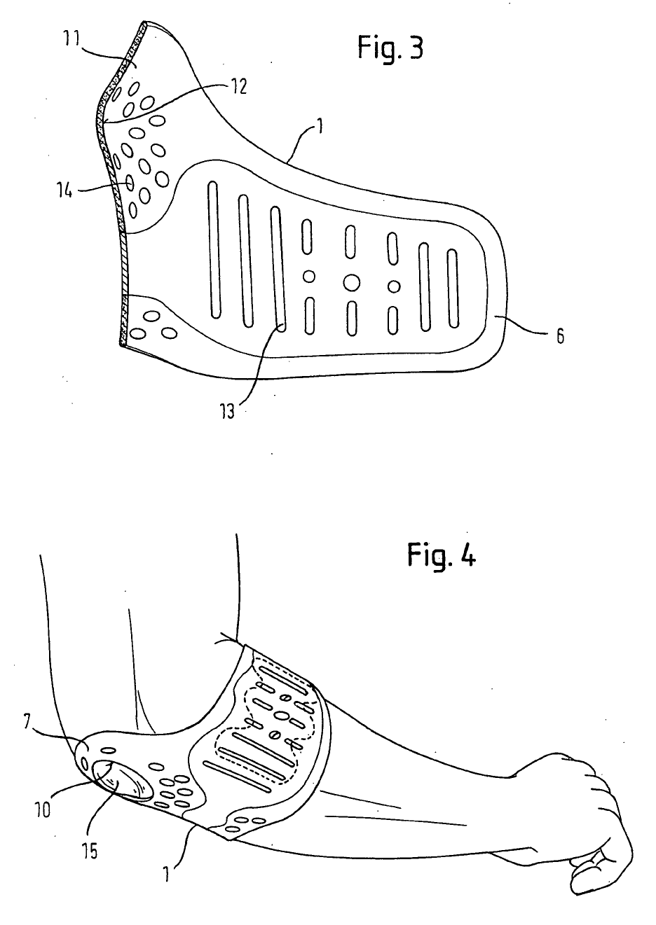

FIG. 3 shows a section along line III-III from FIG. 2;

FIG. 4 shows the epicondylitis clasp fitted to a flexed arm;

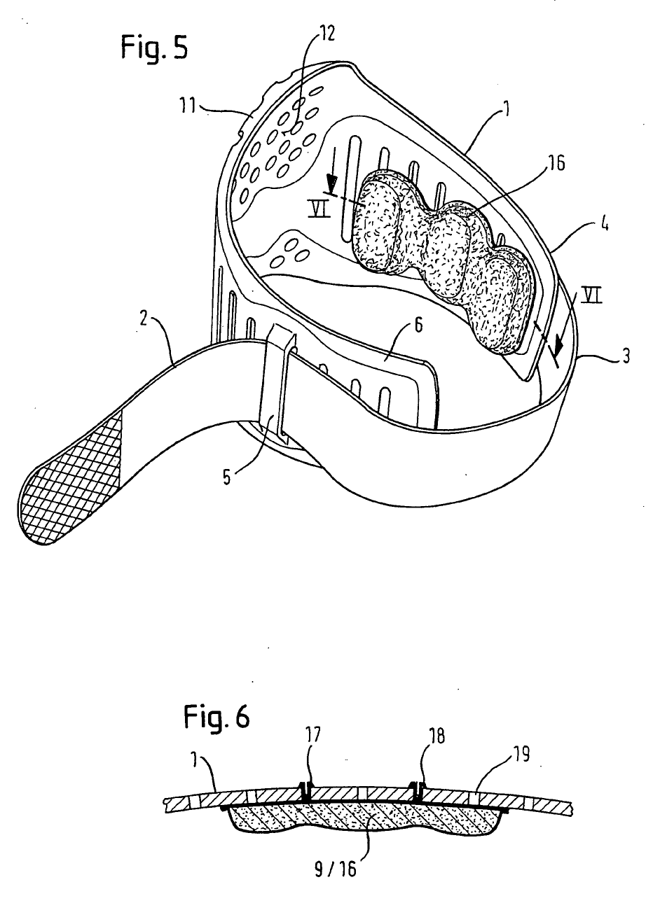

FIG. 5 shows the epicondylitis clasp from FIG. 2 with open securing strap and with a pad attached on the opposite side in relation to FIG. 2; and

FIG. 6 shows a section along line VI-VI from FIG. 5.

The epicondylitis clasp presented in FIG. 1 consists of the holding part 1, which, when fitted to the arm (see FIG. 4), partially encompasses the arm, the holding part 1 leaving free a space between its ends 4 and 6, said space being bridged by the securing strap 2. The securing strap 2 is looped through the oblong eye 5 (see also FIG. 5) at one end 6 of the holding part 1, its outer portion being attached by its one termination 8 to the other termination 3 of the securing strap 2 and being fastened thereto by means of a hook-and-pile fastener. Said termination 3 is likewise fixed to the end 4 of the holding part 1 by means of a hook-and-pile fastener. Said connection between end 4 and termination 3 can also be otherwise accomplished, for example by means of pins which engage offset holes (see, for example, FIG. 6). The holding part 1 can be tightly fitted around an arm by means of suitable tightening of the securing strap 2. Consequently, such tightening of the securing strap 2 is transmitted to the holding part 1, which is firmly pressed against the arm. This method of tightening of the epicondylitis clasp is a known operation.

On its side opposite the two ends 4 and 6 of the holding part 1, the holding part 1 is provided with a tab 7, which forms an arched portion with a hole 10, wherein, when the epicondylitis clasp is fitted to a forearm next to the arm joint, said arched portion encompasses the arm joint from outside and, with the hole 10, positions the tab 7 and, with it, the holding part 1 in the therapeutically required position with respect to the elbow (see also FIG. 4).

The holding part 1 is provided on one of its insides with the pad 9, which, in this case, consists of three juxtaposed interconnected individual pads and in known manner exerts the required pressure on the therapeutically correct area of the arm. As shown in FIG. 1, the epicondylitis clasp can be fitted to either the left or right arm, for which purpose the pad can be suitably attached at opposite points on the inside of the holding part 1.

In FIG. 2, the epicondylitis clasp, which is identical to the one presented in FIG. 1 with respect to its basic construction, is shown with a variant design of the tab 11, which, instead of the hole 10 in FIG. 1 as positioning aid, is provided with a recess 12, which is clearly shown in the sectional drawing in FIG. 3, which presents a section along line III-III from FIG. 2. The recess 12 is slipped protectively over the elbow and thereby fixes the epicondylitis clasp in the therapeutically required position in relation to the elbow joint. With regard to the further design components of the epicondylitis clasp shown in FIG. 2, reference is made to the explanatory remarks in connection with FIG. 1.

FIG. 3 shows a section through the holding part 1 along line III-III from FIG. 2 and clearly presents the recess 12. When the epicondylitis clasp is fitted, the elbow slips, so to speak, into the recess 12, thereby ensuring the correct positioning of the holding part 1 together with the epicondylitis clasp. It should be additionally pointed out that the holding part 1 is provided with a row of slits 13 and openings 14, which guarantee that, with the epicondylitis clasp fitted, there is sufficient access of air to the skin of the arm.

FIG. 4 shows the epicondylitis clasp according to the invention with its holding part 1 fitted next to the elbow joint of an arm, wherein the tab 7 covers the elbow. The hole 10 in the tab 7 accommodates the elbow 15, which passes through the hole 10, thereby fixing the position of the epicondylitis clasp.

FIG. 5 presents the epicondylitis clasp from FIG. 2 with the securing strap 2 in the released position, i.e. with the holding part 1 open. In relation to FIG. 2, the pad 16 is attached on the inside of the holding part 1 in a position which is opposite to the position of the pad 9 in FIG. 2. For this purpose, the pad 9 or 16 is specially connected to the holding part 1, as will be explained below with reference to FIG. 6.

The attachment of the pad 9 or 16 to the holding part 1 is presented in FIG. 6 in a section extending along line VI-VI from FIG. 5. It is apparent therefrom that the pad 9 or 16 is provided with the two locking pins 17 and 18, which are in the form of known slotted spring pins with barbs and each penetrate a hole in the holding part 1. Unoccupied holes are provided with reference character 19. In the position presented in FIG. 6, although the pad 9 or 16 is securely attached to the holding part 1, it can be detached therefrom by pressing together the locking pins 17 and 18, whereupon said locking pins 17 and 18 can be withdrawn through the holes 19, this releasing the pad 9 or 16 from the holding part 1, it then being possible, if necessary, for the pad 9 or 16 to be attached on the opposite side of the holding part 1 in the same manner as shown in FIG. 6. For this purpose, therefore, the two ends 4 and 6 of the holding part 1 are each provided with a plurality of corresponding holes, with the result that the pad 9 or 13 can be attached on either side of the holding part 1, laterally displaced if necessary, as is shown in a comparison by FIGS. 2 and 3.

It should additionally be pointed out that, instead of the locking pins 17 and 18 and the holes 19, the pad 9 or 16 can be provided with a hook-and-pile fastener, which uses a corresponding textile lining on the inside of the holding part 1, this likewise providing a releasable connection between pad 9/16 and holding part 1.

As a result of this optional possibility of attachment of the pad 9/16 on either side of the holding part 1, the epicondylitis clasp according to the invention is suitable both for corresponding treatment of the left or right arm or, for example, of tennis elbow or golfer's arm, wherein, when the epicondylitis clasp is fitted, the tab 7 with its hole 10 or recess 12 ensures that the clasp is always automatically positioned by the elbow of the elbow joint in the correct position and in identical manner, this making it considerably easier for the epicondylitis clasp to be correctly used for treatment in a multiplicity of applications.

1. Epicondylitis clasp, wherein the holding part thereof, encompassing the arm, is tightenable by means of a steplessly adjustable securing strap, the inside of said epicondylitis clasp being provided with a pad for exerting pressure on a pressure-requiring area, said pad being in the form of an oblong pad attached to the holding part, characterized in that a side of the holding part opposite the opening therein—said opening being bridgeable by the securing strap—is provided with a tab, wherein, with the holding part fitted on the arm, said tab covers the elbow joint and, by receiving the elbow, so encompasses the elbow joint that, when the holding part is fitted, the tab, which is guided by the elbow, serves as a positioning aid for positioning the holding part, wherein said holding part can be fitted by means of the positioning aid to either the left or right arm and, for fitting to the left or right arm, is provided on its inside with a support for the pad, by means of which support the pad is removably attached to the holding part by securing means such that the holding part can be fitted on either the left or right arm with the pad in the therapeutically required position.

2. Epicondylitis clasp according to claim 1, characterized in that the tab is provided with a hole to receive the elbow.

3. Epicondylitis clasp according to claim 1, characterized in that the tab is provided with a recess to receive the elbow.

4. Epicondylitis clasp according to claim 1, characterized in that the securing means consist of a hook-and-pile fastener.

5. Epicondylitis clasp according to claim 1, characterized in that the securing means consist of locking pins and holes to receive said locking pins.

6. Epicondylitis clasp according to claim 1, characterized in that the pad is of such length in the circumferential direction of the holding part as to extend beyond the pressure-requiring area.

7. Epicondylitis clasp according to claim 1, characterized in that the pad consists of a plurality of juxtaposed individual pads.

8. Epicondylitis clasp according to claim 1, characterized in that the border of the holding part is softer than the inner region of the holding part.

9. Epicondylitis clasp according to claim 2, characterized in that the securing means consist of a hook-and-pile fastener.

10. Epicondylitis clasp according to claim 3, characterized in that the securing means consist of a hook-and-pile fastener.

11. Epicondylitis clasp according to claim 2, characterized in that the securing means consist of locking pins and holes to receive said locking pins.

12. Epicondylitis clasp according to claim 3, characterized in that the securing means consist of locking pins and holes to receive said locking pins.
